(19) United States
(12) Patent Application Publication
(10) Pub. No.: US 2008/0221514 A1
(43) Pub. Date: Sep. 11, 2008

(54) HEMORRHOID TREATMENT SUPPOSITORY

(76) Inventor: Jiro TAKASHIMA, Houston, TX (US)

Correspondence Address: EGBERT LAW OFFICES, 412 MAIN STREET, 7TH FLOOR, HOUSTON, TX, 77002, US

(21) Appl. No.: 11/682,216

(22) Filed: Mar. 5, 2007

(51) Int. Cl. A61M 1/06

(52) U.S. Cl. 604/75

(57) ABSTRACT

A suppository device has a shaft having a first end and a second end. The first end has a bulbous upper portion. The diameter of the wide section of the bulbous upper portion is between 6 millimeters and 12 millimeters. The shaft has a tapered surface extending toward the second end from the bulbous upper portion. The taper angle of the tapered surface is decreasing to virtually 0 degrees toward the second end. The diameter at the portion of the shaft, separated 4 cm from the wide section of the bulbous upper portion, is between ⅓ and ⅔ of the diameter of the wide section of the bulbous upper portion. The shaft has a length of no less than 4 centimeters.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a hemorrhoid treatment devices. More particularly, the present invention relates to hemorrhoid suppositories that are designed to stay within the anal canal in order to deliver the medication directly to the internal hemorrhoidal area.

2. Description of Related Art Including Information Disclosed Under 37 CFR 1.97 and 37 CFR 1.98

Most hemorrhoid suppositories have a bullet shape. Therefore, it is quite difficult for the bullet-shaped suppositories to stay within the anal canal after they are inserted. Additionally, these bullet-shaped suppositories tend to go upward in the rectum before the medication is applied to the internal hemorrhoid area.

It is object of the present invention is to provide a suppository that can be retained within the anal canal by utilizing the friction between the suppository and the anal canal.

It is another object of the present invention is to provide a suppository that is easy to use, easy to manufacture and relatively inexpensive.

These and other objects and advantages of the present invention will become apparent from a reading of the attached specification and appended claims.

### BRIEF SUMMARY OF THE INVENTION

The present invention is a suppository device that comprises a shaft having a first end and a second end. The first end has a bulbous upper portion. The shaft has a tapered surface extending toward the second end from the bulbous upper portion. The taper angle is decreasing toward second end to virtually a straight surface. The shaft has an average diameter of between 4 millimeters and 10 millimeters inclusive. The shaft has a length of no less than 4 centimeters. The suppository device of the present invention can be formed of a solid material or a material that is dissolvable upon contact with the internal hemorrhoidal area.

### BRIEF DESCRIPTION OF THE SEVERAL VIEWS OF THE DRAWINGS

FIG. 1 is a side elevation view of the suppository device of the present invention.

FIG. 2 is a top view of the suppository device of the present invention.

FIG. 3 is a bottom view of the suppository device of the present invention.

### DETAILED DESCRIPTION OF THE INVENTION

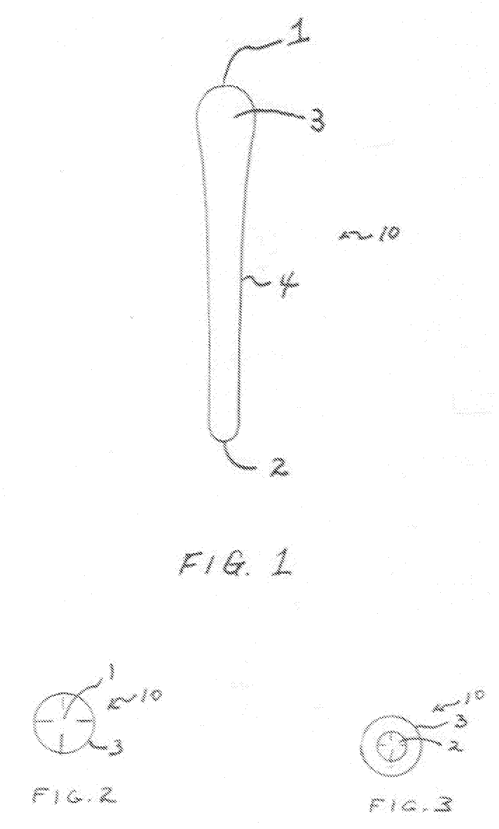

FIG. 1 shows the suppository device 10 in accordance with the teachings of the present invention. The suppository device 10 has a first end 1 and a second end 2. The first end 1 has a bulbous upper portion 3. A tapered surface 4 extends from the bulbous upper portion 3 toward the second end 2.

As can be seen in FIG. 1, the angle of the tapered surface 4 decreases from the bulbous upper portion 3 toward the second end 2. Approximately between one-third and one-half of the portion of the tapered surface 4 which extends to the second end 2 is virtually a straight surface. The diameter of the wide section 5 of the bulbous upper portion 3 is approximately between 6 and 12 millimeters and the diameter of the portion of the shaft separated approximately 4 cm from the wide section 5 of the bulbous upper portion 3 is between ⅓ and ⅔ of the wide section 5 of the bulbous upper portion 3. The average diameter of the device 10 is between 4 millimeters and 8 10 millimeters inclusive.

When the device 10 is inserted into the anal canal, the outward peristaltic motion within the anal canal will start to remove the device to the outside of the anus. Since the device 10 has a slight tapered surface to resist this outward peristaltic motion, the device 10 will stay within the anal canal.

FIG. 2 illustrates a top view of the device 10. In FIG. 2, it can be seen that the cross section of the device is round. The upper end 1 is particularly illustrated. The upper end will curve outwardly toward the bulbous upper portion 3.

FIG. 3 is a bottom view of the device 10. In FIG. 3, it can be seen that the second end 2 slightly tapers outwardly toward the bulbous upper portion 3.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

1. A suppository device comprising:
a shaft having a first end and a second end, said first end having a bulbous upper portion, said shaft having a tapered surface extending toward said second end from said bulbous upper portion, taper angle of said tapered surface decreasing to a virtually 0° degree toward said second end, said bulbous upper portion have a widest section with a diameter between 6 and 12 millimeters inclusive, said shaft having a section with a diameter, said diameter of said section of said shaft being separated 4 cm from said widest section of said bulbous upper portion and being approximately between ⅓ and ⅔ of said diameter of said widest section of said bulbous upper portion, said shaft having a length of no less than 4 centimeters.
